(19) United States
(12) Patent Application Publication
(10) Pub. No.: US 2025/0204975 A1
(43) Pub. Date: Jun. 26, 2025

(54) ELECTROSURGICAL DEVICE AND METHOD OF USE

(71) Applicant: Meditrina, Inc., San Jose, CA (US)

(72) Inventors: Zsolt TRUCKAI, San Jose, CA (US); Tamas J. TRUCKAI, San Jose, CA (US); John H. SHADDUCK, San Jose, CA (US); Csaba TRUCKAI, San Jose, CA (US)

(73) Assignee: Meditrina, Inc., San Jose, CA (US)

(21) Appl. No.: 18/390,702

(22) Filed: Dec. 20, 2023

(51) Int. Cl. A61B 18/14; A61B 18/00

(52) U.S. Cl. CPC A61B 18/1482; A61B 2018/00083; A61B 2018/00208; A61B 2018/00601; A61B 2018/1407; A61B 2018/1475

(57) ABSTRACT

Bipolar electrosurgical devices and methods of use using loop electrodes for cutting and resecting tissue, for example, in endoscopic urology and gynecology procedures.

### FIELD OF THE INVENTION

The present invention relates to bipolar electrosurgical devices and methods of use for cutting tissue, for example, in endoscopic urology and gynecology procedures.

### SUMMARY OF THE INVENTION

Some aspects in accordance with principles of the present disclosure relate to a bipolar electrosurgical device. The electrosurgical device can comprise a handle and a small diameter, elongated shaft adapted for introduction through a working channel of an endoscope. The working end of the device has a form of loop electrode that is extendable outward from the shaft, a selected dimension for cutting tissue at a selected depth.

For example, a variation of an electrosurgical device include an electrosurgical resection device including: a handle coupled to an introducer member extending about a central axis to a working end, wherein the introducer member has an outer surface that defines an introducer envelope; an electrode shaft, an RF loop electrode carried by the electrode shaft and positioned at the working end extending in a transverse plane that is from 0° to 45° transverse to the central axis; a drive assembly, or deployment mechanism) within the handle and coupled to the electrode shaft, where the drive assembly is configured to be from the handle configured to move the electrode shaft to move the RF loop electrode between a first position within the introducer envelope for introduction into a working space and a second position radially outward from the introducer envelope for resecting tissue.

Variations of the device and method include a RF loop electrode that is carried at a distal end of an insulative shaft extending through a central passageway in the introducer member,

Additional variations of the device and method can include a deployment mechanism that is adapted to move the RF loop electrode between the first position and the second position by at least one of rotation of the insulative shaft and lateral movement of the insulative shaft.

Variations of the deployment mechanism can be further adapted to move the RF loop electrode axially between a distal position and a proximal position in the introducer member.

The deployment mechanism can be actuated by at least one of a motor drive and manual actuation.

Variations of the RF loop electrode can include an inflexible wire, or any similar structure.

In any variation, the RF loop electrode, when in the second position, can be positioned outward of the introducer envelope by at least 1 mm, 2 mm, 4 mm, or any distance therebetween. Unless specifically recited in the claims, the RF loop electrode can be positioned outward of the introducer envelope by any distance.

In some variations, the present disclosure includes an electrosurgical device further including an RF source coupled to the RF loop electrode, a negative pressure source communicating with the central passageway in the introducer member, a motor drive operatively coupled to the deployment mechanism, and a controller configured to control operating parameters of the RF source, negative pressure source and motor drive.

Variations of the devices can include including one or more accelerometers carried by the electrosurgical device on any portion of the device, and configured to send signals to the controller relating to movement of the electrosurgical device.

In some variations, the present disclosure includes an electrosurgical device wherein the introducer member includes an articulating section in the working end that is actuatable from the handle.

In some variations, the present disclosure includes an electrosurgical device wherein the RF loop electrode includes an active electrode and further including a return electrode carried in the working end.

In some variations, the present disclosure includes a method of resecting tissue in a working space in a patient, including: providing an introducer having an outer surface that defines an introducer envelope, the introducer extending about a central axis to a working end carrying an RF loop electrode that is moveable between a first position within the introducer envelope and a second position radially outward from a surface of the introducer that defines a selected cutting depth; introducing the working end in the first position through a working channel of an endoscope into the working space; moving the RF loop electrode to the second position; activating the RF loop electrode and translating the RF loop electrode through tissue at the selected cutting depth to thereby cut a tissue strip; and moving the RF loop electrode from the second position to the first position thereby mobilizing the tissue strip.

Variations of the devices in the present disclosure include a method wherein the RF loop electrode extends in transverse plane that is from 0° to 45° transverse to a central axis of the introducer in the second position.

Variations of the methods include selecting the cutting depth by a mechanism in a handle portion of the introducer.

The methods and devices described herein can include an RF loop electrode that is moved between the first position and the second position in a rotational direction relative to the central axis of the introducer. Alternatively, or in combination, the RF loop electrode is moved between the first position and the second position in a transverse direction relative to the central axis of the introducer.

The RF loop electrode can be moved between the first position and the second position by at least one of a motor drive and manual actuation.

In some variations, the present disclosure includes a method wherein the RF loop electrode is translated through tissue by at least one of a motor drive and manual actuation.

In some variations, the present disclosure includes a method wherein a controller is configured to control movement of the RF loop electrode.

In some variations, the present disclosure includes a method sending signals of movement of the introducer or RF loop electrode to the controller using an accelerometer, controlling movement of the RF loop electrode responsive to the signals using the controller.

### BRIEF DESCRIPTION OF THE FIGURES

FIG. 1 is a perspective view of an RF resecting device corresponding to the invention that has a handle connected to an elongate introducer assembly with a bi-polar loop electrode that can be rotatably extended and retraced from the introducer together with a block diagram of a negative pressure source, RF source, and controller operatively coupled to the device.

FIG. 2 is an enlarged perspective cut-away view of the working end of the RF device of FIG. 1.

FIG. 3A is a perspective view of the working end of the RF device of FIGS. 1 and 2 showing the RF loop electrode in a first retracted position relative to the introducer member of the device.

FIG. 3B is another view of the working end of FIG. 3A showing the RF loop electrode rotated to a position outward from the introducer to provide a first selected cutting depth.

FIG. 3C is another view of the working end of FIGS. 3A-3B showing the RF loop electrode rotated outward further to provide a second selected cutting depth.

FIG. 3D is a view of the working end of FIGS. 3A-3C showing the RF loop electrode rotated outward further to provide a third selected cutting depth.

FIG. 4 is a partly sectional side view of the working end of the device of FIGS. 1 to 3D showing the loop electrode rotatable in a transverse plane relative to the introducer axis.

FIG. 5 is a side view of another variation of a working end showing an RF loop electrode that is rotatable outward from an introducer, with the form of the electrode defining a non-transverse plane relative to the introducer axis.

FIG. 6 is a perspective view of another variation of a working end of an RF device wherein the RF loop electrode is rotatable outward from a slot in the introducer.

FIG. 7A is a schematic view of an initial step in the method of use for resecting a bladder tumor wherein an endoscope is introduced into a patient's bladder, followed by advancing the RF resecting device of FIG. 1 through a working channel of the endoscope, articulating the working end of the RF device and rotating the loop electrode outward from the introducer to provide a selected cutting depth.

FIG. 7B is an enlarged view of a subsequent step of the method of use wherein the RF loop electrode is energized, and the physician cuts a path into the targeted tissue with a margin around the tumor.

FIG. 7C is a view of another subsequent step of the method wherein the energized RF loop electrode cuts a path under the tumor as the physician moves the working end.

FIG. 7D is a view of the final step of the method wherein the energized RF loop electrode has cut a path fully under the tumor, and the physician rotates the energized loop electrode outwardly to cut, resect and mobilize the tissue chip.

FIG. 8 is a perspective view of another variation of an RF resecting device with a handle connected to an elongate introducer assembly wherein a bi-polar loop electrode is configured for lateral movement outward from the introducer using a cam mechanism together with a block diagram of a negative pressure source, RF source, electrical source, and controller operatively coupled to the device.

FIG. 9 is an exploded view of the working end of the RF device of FIG. 8, showing the RF loop electrode assembly and components of the cam mechanism for deploying the RF loop electrode.

FIG. 10A is a transparent perspective view of the working end of FIGS. 8 and 9 with the RF loop electrode in a retracted position.

FIG. 10B is a transparent view of the working end of FIG. 10A with the RF loop electrode in a laterally extended position.

FIG. 11A is a sectional view of the working end of FIGS. 8 and 9 with the RF loop electrode in a retracted position.

FIG. 11B is a sectional view of the working end of FIG. 11A with the RF loop electrode in a partially extended position outward from the introducer.

FIG. 11C is a sectional view of the working end of FIGS. 11A-11B with the RF loop electrode in a fully extended position outward from the introducer.

FIG. 12 is a perspective view of another variation of an RF resecting device with an elongate introducer carrying a bi-polar loop electrode together and an accelerometer in the handle coupled to a controller and motor drive for moving the loop electrode in response to signals from the accelerometer.

FIG. 13A is a schematic view of an initial step in an endoscopic method of using the resecting device of FIG. 12 in a fibroid resection procedure.

FIG. 13B is a subsequent step of the method of FIG. 13A showing advancement of the resecting device through a working channel of an endoscope and rotation outward of the loop electrode from the introducer member to a cutting position.

FIG. 13C is an enlarged view of a subsequent step of the method wherein the energized RF loop electrode cuts a path into tissue as the physician moves the working end.

FIG. 13D is the final step of the method wherein the energized RF loop electrode has cut a path under a strip of tissue, and the accelerometer signals the motor drive to rotate the loop electrode to fully resect and mobilize the tissue strip.

FIG. 14 is a perspective view of another variation of an RF resecting device with a deployable bi-polar RF loop electrode deployable from a window in the introducer member wherein a motor drive is configured to move the loop electrode outward, inward, and axially in the window.

FIG. 15A is a schematic view of an initial step in using the resecting device of FIG. 14, preparing to cut into tissue with a deployed, energized RF loop electrode.

FIG. 15B is a subsequent step of the method of FIG. 15A, wherein the energized RF loop electrode is motor-driven to cut an elongated path in tissue as the physician holds the working end in a stable position.

FIG. 15C is a subsequent step of the method of FIGS. 15A-15B, wherein the motor drive rotates the energized RF loop electrode to fully resect a strip of tissue and then return the de-energized loop electrode to a default position.

FIG. 16 is a perspective view of another variation of an RF resecting device with a working end carrying a deployable bi-polar loop electrode wherein the working end is motor-driven to axially reciprocate in an outer sleeve coupled to a handle.

FIG. 17 is a perspective view of another variation of an RF resecting device with a working end carrying a deployable bi-polar loop electrode wherein the introducer member comprises an extrusion of a polymeric material partly surrounded by a thin-wall metal sleeve.

FIG. 18 is an exploded view of the components of working end of the device of FIG. 17.

FIG. 19 is an enlarged perspective view of a working end similar to that of FIG. 17.

FIG. 20 is a perspective view of a variation of an RF loop electrode.

FIG. 21 is an end view of a working end similar to that of FIG. 19.

FIG. 22 is a cut-away view of the working end of FIG. 21

### DETAILED DESCRIPTION

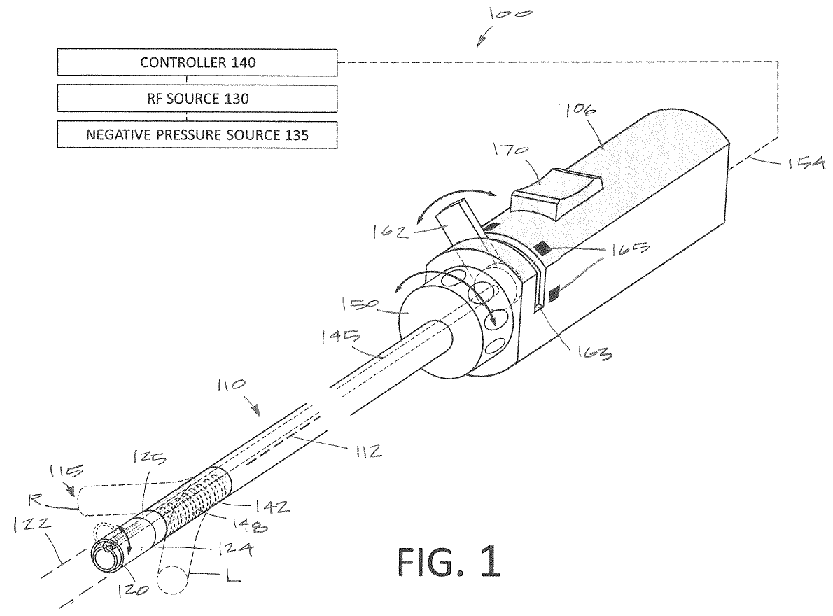

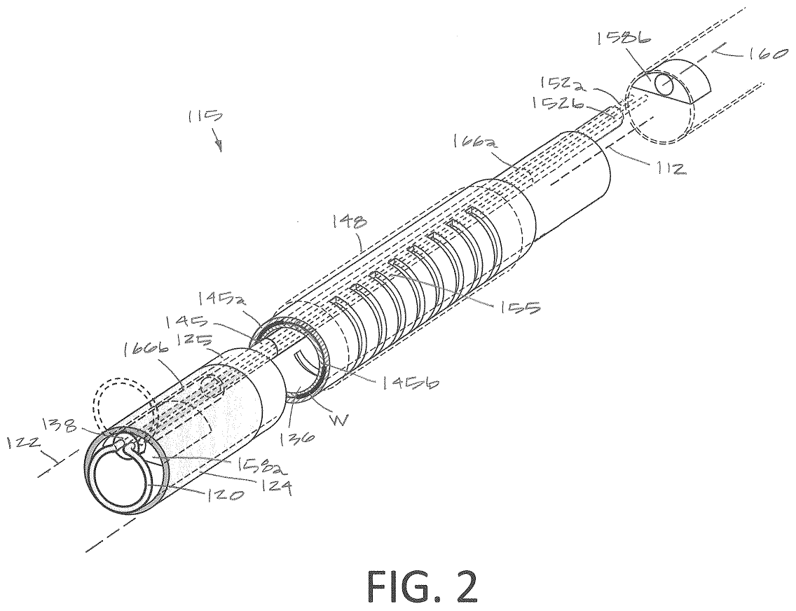

FIGS. 1 and 2 illustrate an electrosurgical tissue resecting device 100 comprising a handle 106 coupled to an elongated introducer member 110 extending about central axis 112 and having a diameter of 3 mm to 10 mm. The length of the introducer 110 can be highly elongated and suited for introduction through the working channel of an endoscope 114 (see FIG. 5A). In a variation, the introducer member 110 has a working end 115 shown in FIG. 2 carrying an RF active loop electrode 120 that is extendable outwardly by a deployment mechanism to be positioned outward of the cylindrical introducer envelope 122 of the introducer member 110. A distal portion of the working end 115 has an electrically insulative coating 124, with the adjacent proximal portion comprising a return electrode 125.

In the variation of FIG. 1, the RF resecting device 100 is operatively connected to a remote RF source 130 that is coupled to the active and return electrodes 120 and 125, a negative pressure source 135 that communicates with an interior passageway 136 in the introducer 110 (FIG. 2). The controller 140 is adapted to control the operating parameters of the RF source 130 and the negative pressure source 135.

The variation of FIG. 1 has an introducer member 110 with an articulating or deflecting section 142 in the working end 115, shown in more detail in FIG. 2. Typically, the deflectable section comprises an assembly of co-axial thin-wall slotted sleeves with outer sleeve 144a and inner sleeve 144b shown in the cut-away view of FIG. 2. Such slotted sleeve assemblies are known in the art, for example, as shown in U.S. Pat. No. 10,058,336 by Truckai et al., issued Aug. 28, 2018. The slotted sleeves 144a and 144b are fixed to one another with weld W (FIG. 2) at the distal end of the assembly, and the inner slotted sleeve 144b can be moved axially relative to the outer slotted sleeve 144a to deflect the deflectable section 142 as shown in dashed lines in FIG. 1 between left and right deflections indicated at L and R. A thin-wall flexible polymeric sleeve 148 encases the slotted sleeve assembly and is shown as a dashed line in FIG. 2. In this variation, a rotatable hub 150 at the proximal end of the shaft 110 adjacent the handle 106 is rotated clockwise or counter-clockwise to deflect the working end 115 in the left and right deflections, L and R, as shown in FIG. 1. The rotatable hub 150 is operatively coupled a proximal end to the inner sleeve 144b within the handle 106 with a helical coupling so that rotation of the hub 150 moves the inner sleeve axially relative to axis 112 in either the proximal or distal direction to deflect the working end as shown in FIG. 1.

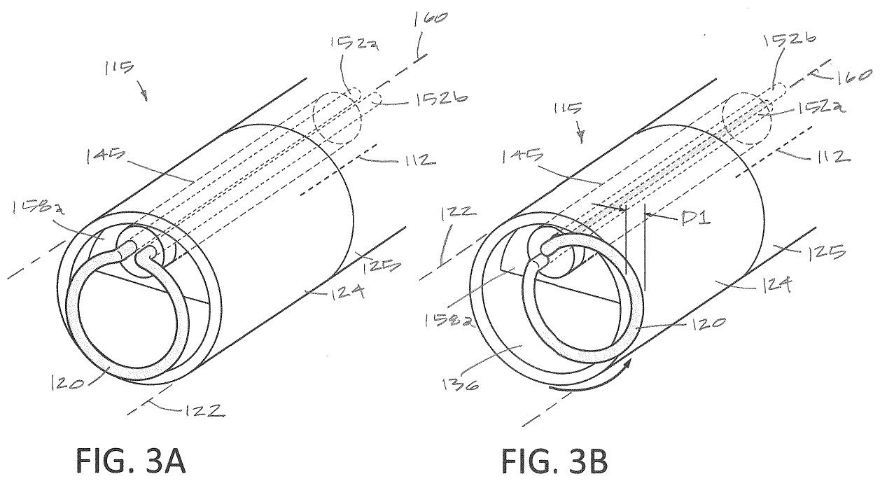

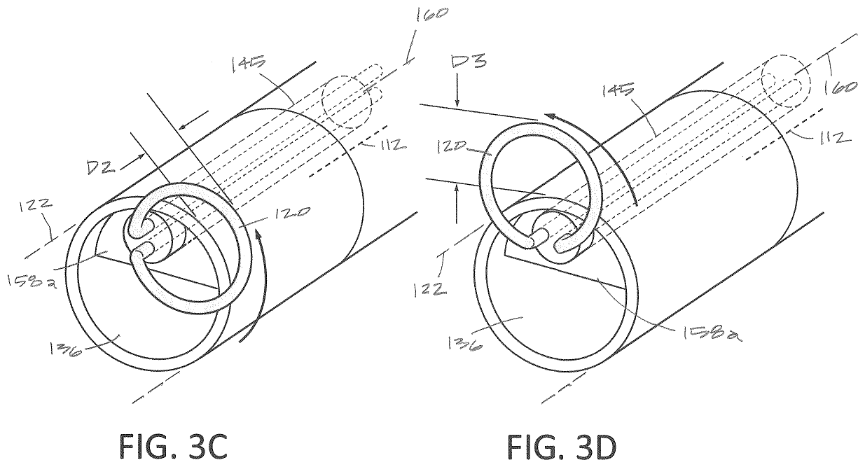

FIG. 2 further illustrates the components coupled to the moveable loop electrode 120 carried by the working end 115. The active loop electrode 120 extends distally from the distal end 138 of a rotatable, electrically insulated electrode shaft 145 that is carried within the interior passageway 136 of the introducer member 110. The electrode shaft 145 extends entirely through the passageway 136 to the handle 106 and is rotatable by a rotation mechanism that is further described below. The loop electrode 120 has first and second electrical leads 152a and 152b that extend through the electrode shaft 145 to the handle 106 and are coupled by an electrical cable 154 to the RF source 130 (FIG. 1). The electrode shaft 145 is configured with a flexible section 155 extending through the deflecting portion 142 of the introducer 110 that comprises that a flexible drive shaft as known in the art. Such a flexible drive shaft can comprise a helical spring member, as is known in the art of flexible dive shafts. The electrical leads 152a, 152b within the flexible drive shaft portion 155 are small diameter insulated wires and are flexible. The electrode shaft 145 is rotatable within a plurality of collars fixed in the walls of introducer member 110 with collars 158a and 158b shown in FIG. 2. A plurality of such collars may be spaced apart in the passageway 136. The rigid portions of the introducer member 110 may have a continuous bore that carries the electrode shaft 145 in a wall of the inner sleeve 144b. As can be understood from FIGS. 1 and 2, the rotatable electrode shaft 145 has an off-center axis 160 that is offset from and parallel to the central axis 112 of the introducer member 110. Thus, it can be understood that rotation of the off-center electrode shaft 145 around axis 160 will rotate the loop electrode 120 from a first position within the cylindrical introducer envelope 122 of the introducer member 110 to selected other positions where the loop electrode 120 is positioned outward of the cylindrical introducer envelope 122 as shown in FIGS. 2 and 3B-3D. In FIG. 1, it can be seen that an actuator arm 162 is coupled to the electrode shaft 145 and is moveable in an arc in slot 163 in the handle to move the loop electrode 120 outwardly and inwardly. As can be seen in FIG. the handle 106 can include markings 165 near the slot 163, indicating the position of the electrode 120. An actuator or power button 170 is provided in the handle 106 to energize the RF loop electrode 120 as described below in a method of use.

Referring again to FIG. 2, the distal portion of the outer sleeve 144a has the electrically insulative coating 124 that can comprise a polymeric or ceramic layer. In another variation, the distal portion of the outer sleeve 144a can be dipped in an epoxy to provide the insulative coating 124. The insulative coating 124 is adapted to space apart the RF loop electrode 120 from the return electrode 125, which can comprise exposed portions of the outer sleeve 144a. In FIG. 2, it also can be seen that the inner sleeve 144b may only extend within the outer sleeve 144a distally beyond the deflecting section 142 to the region of weld 144. Thus, the distalmost section of working end 115 would comprise only the outer sleeve 144a. Accordingly, the diameter of the electrode shaft 145 may have a smaller diameter portion 166a in the introducer section having both outer and inner sleeves 144a, 144b and a slightly large diameter portion 166b in the distalmost section of the working end 115 that comprises only the outer sleeve 144a.

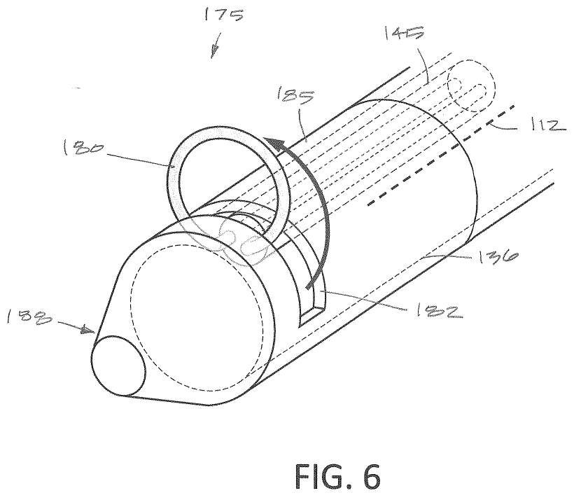

FIGS. 3A-3D are enlarged views of the working end 115 of the device 100 with the loop electrode 120 in various deployed positions with FIGS. 3B top 3D showing the electrode rotated outwardly from the introducer member 110 by the rotatable deployment mechanism. In FIG. 3A, the loop electrode 120 is in a first 0° position or introducer position where the electrode 120 does not extend outside the envelope 122 of the introducer and thus is configured for introduction through the working channel of an endoscope 114 (FIG. 6A). FIG. 3B shows the electrode shaft 145 after being rotated approximately 60° wherein the RF loop electrode 120 at its maximum outward point now extends outwardly of the introducer surface a dimension indicated as D1, which can be from 1 mm to 3 mm in this variation. Now, turning to FIG. 3C, the electrode shaft 145 is shown after being rotated by about 120° from the initial 0° position, where the depth of cutting depth of the RF loop electrode 120 is indicated at D2, which can be from 2 mm to 5 mm in this variation. FIG. 3D shows electrode shaft 145 after being rotated 180° from the initial 0° position, where the cutting depth of the RF loop electrode 120 is indicated at D3, which can be from 3 mm to 6 mm in this variation.

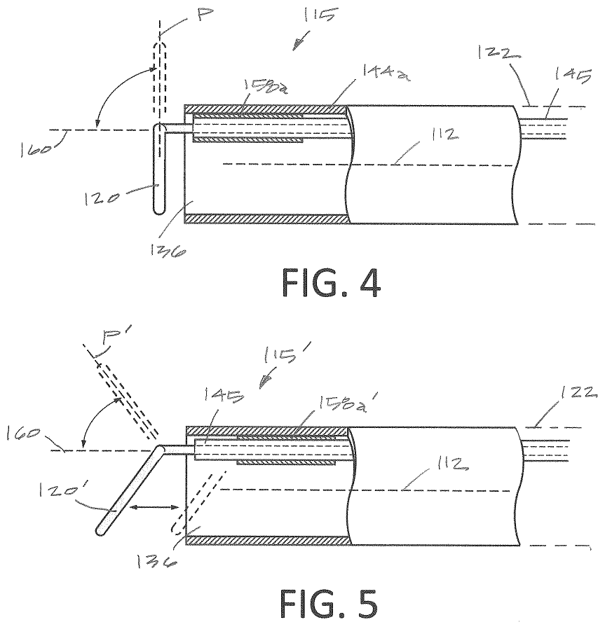

FIG. 4 is a side view of the working end 115 of FIGS. 2 and 3A-3D showing the RF loop electrode 120 angled from the electrode shaft 145 in a transverse direction or 90° direction from the axis 160 of the electrode shaft. When rotated as shown in FIGS. 3B-3D, the RF loop electrode 120 in all resecting positions rotates in a transverse plane P, as shown in FIG. 4.

FIG. 5 is a side view of a variation of a working end 115′ that has an RF loop electrode 120′ extending in plane P′ away from the electrode shaft 145 in a non-transverse angle in a range between 45° and 90° away from axis 160 of the electrode shaft 145. Thus, the scope of the invention and method of the invention includes an RF loop electrode that can be moved outward from the introducer envelope 122 and define a transverse plane P (FIG. 4) or a non-transverse plane P′ as in FIG. 5 that is less than 90° but more than 45° relative to axis 160 of the electrode shaft 145 or the parallel axis 112 of the introducer member 110. In FIG. 5, the loop electrode 120′ also is extendable and retractable in the interior passageway 136 in the first non-rotated position with collar 158a′ moved proximally in the passageway 136.

FIG. 6 illustrates another variation of a working end 175 that is similar to the working end 115 of FIGS. 1 to 4 described previously, except the variation of FIG. 6 is configured with a loop electrode 180 that rotates out of a slot 182 in an outer sleeve 185 that has a closed tip 188. In all other respects, the variation of FIG. 6 operates the same as the variation of FIGS. 1 to 4.

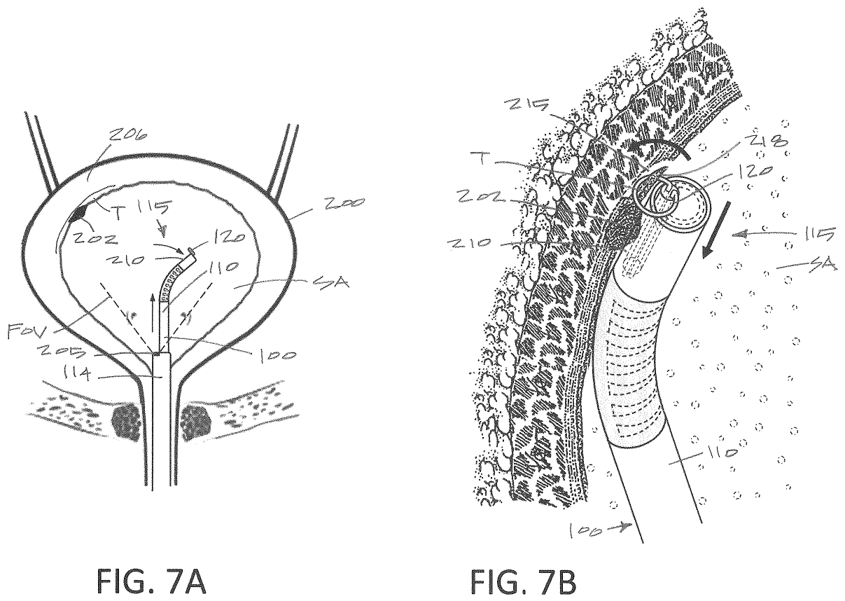

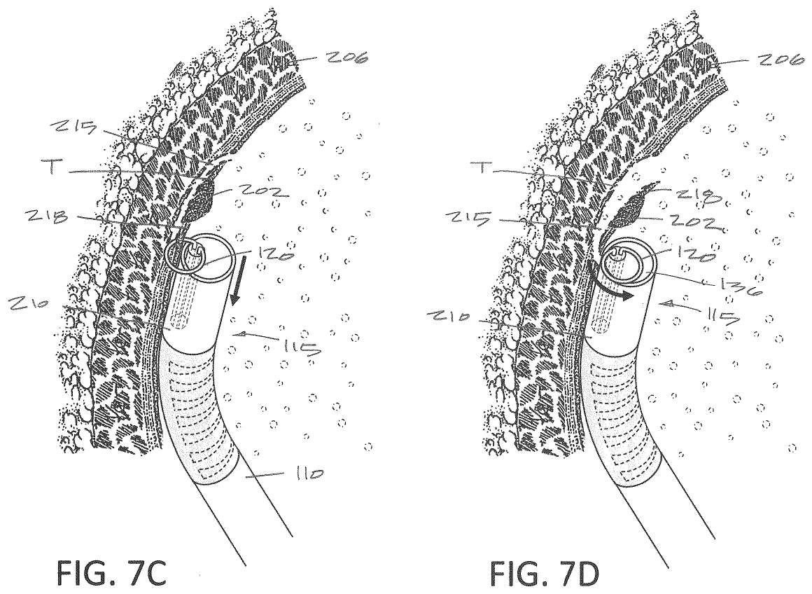

Now, turning to FIGS. 7A-7D, a method of the invention, is shown in an exemplary procedure of introducing the resecting device 100 of FIGS. 1 to 4 into a patient's bladder 200 to resect a bladder tumor 202. FIG. 7A shows an endoscope 114 being introduced into the patient's bladder 200 with saline SA introduced through the endoscope to distend the bladder as is known in the art. The introducer member 110 of the tissue resecting device 100 is introduced through the working channel of endoscope 114, wherein the field of view FOV of the image sensor 205 allows for viewing targeted tissue T of the bladder wall 206 and the bladder tumor 202. FIG. 7A further shows the working end 115 of the resection device 100 being articulated and the RF loop electrode 120 being moved into a cutting position outward of the outer surface 210 of the articulated introducer member 110, which can be any selected cutting depth as shown in FIGS. 3B to 3D that is determined by observations of the tumor 202 by the physician. The working end 115 is articulated to an angled position that is suited for alignment with the portion of the bladder wall 206 that has the tumor 202. The articulation of the working end 115 and the outward movement of the loop electrode 120 are accomplished by the physician moving the actuators in the handle 106 as described above (FIGS. 1 and 2).

As an example. FIG. 7B shows that the physician has selected a cutting depth as in FIGS. 3B to 3D, and the cutting depth is approximately the estimated depth of the tumor 202. In FIG. 7B, the physician then activates the RF loop electrode 120 by pressing power button 170 in the handle (FIG. 1) and cuts a path 215 into the bladder wall 206 near the tumor 202 until the surface 210 of the introducer member 110 is pressed against the bladder wall. In FIG. 7B, it can be understood that the physician translates the working end 115 in a proximal direction over the targeted tissue T, which includes a margin around the tumor 202 wherein the loop electrode 120 starts to cut a tissue chip 218 at the selected depth.

FIG. 7C illustrates a subsequent step wherein the physician translates the working end 115 further over the targeted tissue T with the loop electrode 120 cutting through or under the tumor 202 to further cut the tissue chip 218 and provide a margin on the opposing side of the tumor. Thus, in FIG. 7C, the physician has cut a path 215 completely under the bladder tumor 202. FIG. 7D then shows the physician rotating the RF loop electrode 120 from the outward cutting position toward the 0° position of FIG. 3A to thereby cut off the tissue chip 218 so that the tissue chip is entirely mobilized. The tissue chip 218 then may be extracted from the bladder 200 by suction through the interior passageway 136 in the introducer member 110 or by other means.

In FIGS. 7B to 7D, the resection of such a bladder tumor 202 is shown in a single cut with the RF loop electrode 120. It should be appreciated that in a typical procedure, the steps of the method shown in FIGS. 7B-7D can be repeated a number of times with suitable lesser cutting depths to resect a tumor with suitable margins.

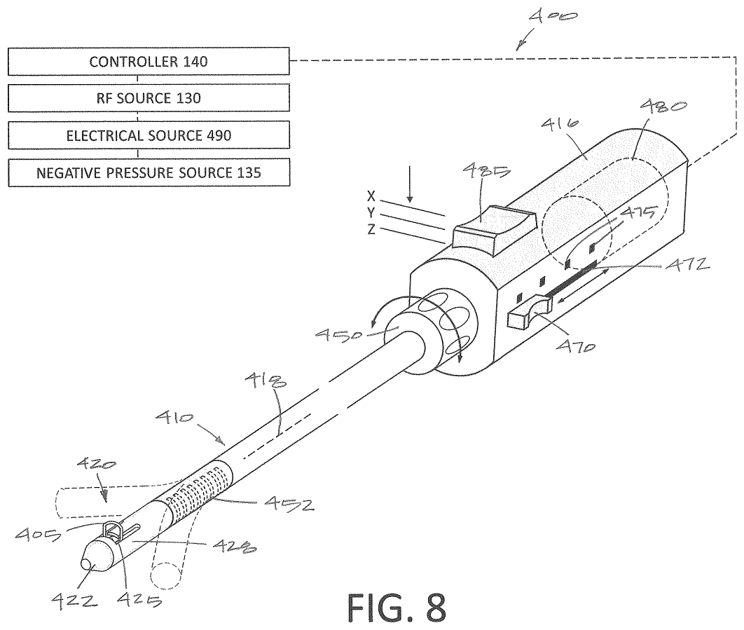

Now, turning to FIG. 8, another variation of a resecting device 400 is shown, which is similar to the device 100 of FIGS. 1 and 2, except the resecting device 400 has an RF loop electrode 405 that can be actuated to move outward from the introducer member 410 in a transverse direction by means of a cam mechanism described below. In FIG. 8, it can be seen that the resecting device 400 has a handle 416 coupled to the elongate introducer member 410 extending about longitudinal axis 418 to a working end 420. The working end 420 has a closed tip 422 as in the variation of FIG. 6 but could have an open end as in the variation of FIGS. 1 and 2. The RF active electrode 405 is movable outward from a partly circumferential slot 425 in the outer sleeve 428 of the working end 420. The resecting device 400 again is coupled to RF source 130, negative pressure source 135, and controller 140, as described above.

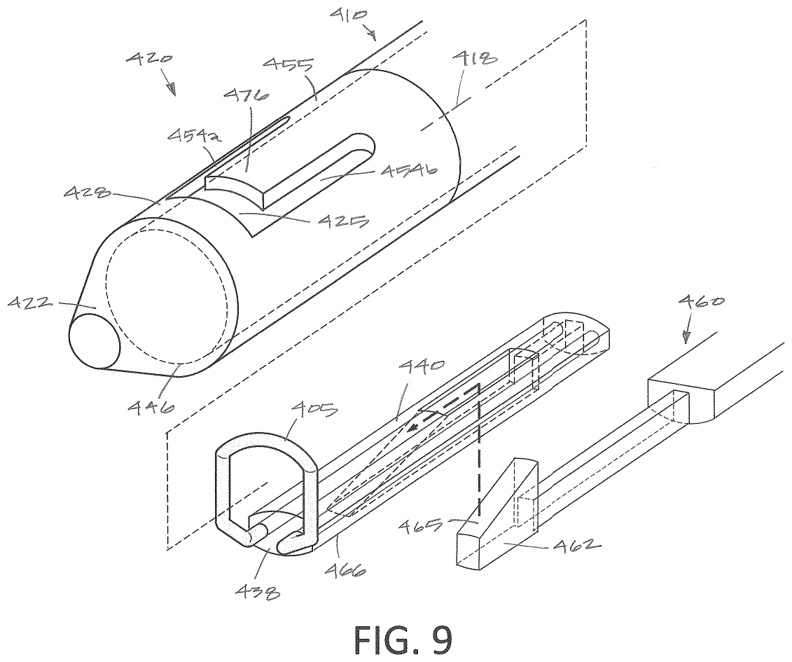

FIG. 9 is an exploded and partly transparent view of a distal portion of the introducer member 410, showing components of the cam mechanism that is adapted for moving the RF loop electrode 405 inward and outward from the introducer member 410. In FIG. 9, it can be seen that the RF loop electrode 405 is carried at the distal end 438 of an electrode shaft 440 that extends through a passageway 446 in the introducer member 410 to the handle 416 (FIG. 8). The introducer member 410 has a rotatable hub 450 that is adapted for articulating the articulating section 452 (FIG. 8) as described in the previous variation.

FIG. 9 shows that the outer sleeve 428 of the introducer member 410 is configured with the partly circumferential slot 425 from which the RF loop electrode 405 can be extended outwardly and retracted inwardly. The introducer member 410 further has axial slots 454a and 454b that are dimensioned to receive axial movement of the RF loop electrode 405 when the electrode has been moved to an outward cutting position. The distal section of the outer sleeve 428 has an insulative coating 455, as described in the previous variation.

In FIG. 9, it can be seen that a cam mechanism comprises an elongated shaft 460 that extends from the handle 416 to a distal wedge-shaped member 462 with an angled cam surface 465. The wedge-shaped member 462 is adapted to slide axially in a slot in the electrode shaft 440. It can be understood that when the wedge-shaped member 462 with its angled cam surface 465 is moved in the distal direction, the cam surface 465 will contact the adjacent surface 466 of electrode shaft 440 and move the loop electrode 405 transverse relative to the axis 418 of the introducer member 410. In FIG. 8, it can be seen that a slider grip 470 in the handle 416 is coupled to the elongated shaft 460 and is adapted to slide in slot 472 in the handle 416 to move the cam surface 465 distally to thereby move the RF loop electrode 405 outwardly from the introducer member 410. The handle 416 further includes indicators 475 at various positions of the slider grip 470, which correspond to different cutting depths of the RF loop electrode 405 as it extends outwardly from the surface 476 of the introducer member 410.

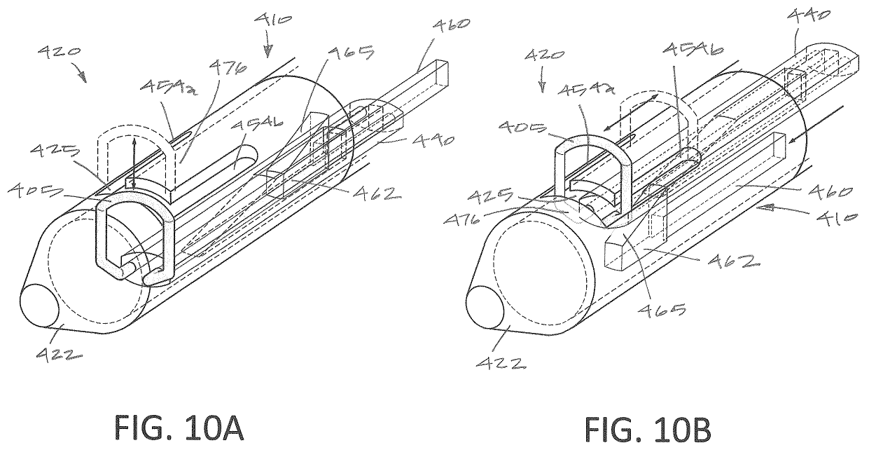

Now, turning to FIG. 10A, a transparent view of the working end 420 is shown with the elongate shaft 460 and cam surface 465 in a proximal position with the electrode shaft 440 and RF loop electrode 405 in a corresponding retracted position within the envelope of the introducer member 410. FIG. 10B shows the elongate shaft 460 and cam surface 465 in a distal extended position, which moves the electrode shaft 440 upwardly and the loop electrode 405 outwardly from the slot 425 in the introducer member 410. It can be understood that the loop electrode 405 can be extended outwardly from slot 425 in any selected dimension and maintained in such a position during use for any selected cutting depth.

Another aspect of the invention is shown in FIGS. 8 and 10B and comprises a motor drive 480 carried in handle 416 that is adapted to reciprocate the assembly of the electrode shaft 440 and the elongate shaft 460 carrying the cam surface 465. As can be seen in FIG. 10B, the loop electrode 405 can be reciprocated in axial slots 454a and 454b in the introducer member 410. Thus, a method of use allows the physician (i) to articulate the working end 420 as needed for a procedure, (ii) then energize the RF loop electrode 405 and press it into targeted tissue to cut a path, (iii) then maintain the working end 420 in a fixed location pressed against the targeted tissue, and (iv) then actuate the motor drive 480 to reciprocate the loop electrode 405 back and forth to cut an underlying path in the targeted tissue similar to that shown in FIGS. 6B and 6C. Thereafter, the physician can lift the loop electrode 405 away from the targeted tissue with the RF electrode energized to mobilize the tissue chip.

In the variation of FIGS. 8 to 10B, the device 400 can be actuated stepwise by first pressing the power button 485 (FIG. 8) from position X to Y, which energizes the RF loop electrode 405 and then can cut into the targeted tissue as described above. Next, the physician can press the power button 485 from position Y to position Z, which will reciprocate the energized loop electrode 405 in a single stroke back and forth. The physician can then release the power button 485 from position Z to position Y, and with the RF electrode 405 still energized, the physician can lift the working end 420 away from the tissue to cut the end of the tissue chip. In other variations, the controller 140 may reciprocate the loop electrode 405 back and forth a selected number of times to cut tissue. As can be seen in FIG. 8, the controller 140 also is coupled to an electrical source 490 that energizes the motor drive 480 in the handle 416. Thus, the controller 140 controls the RF source 130, the negative pressure source 135 as described previously, as well as the operating parameters of the motor drive 480.

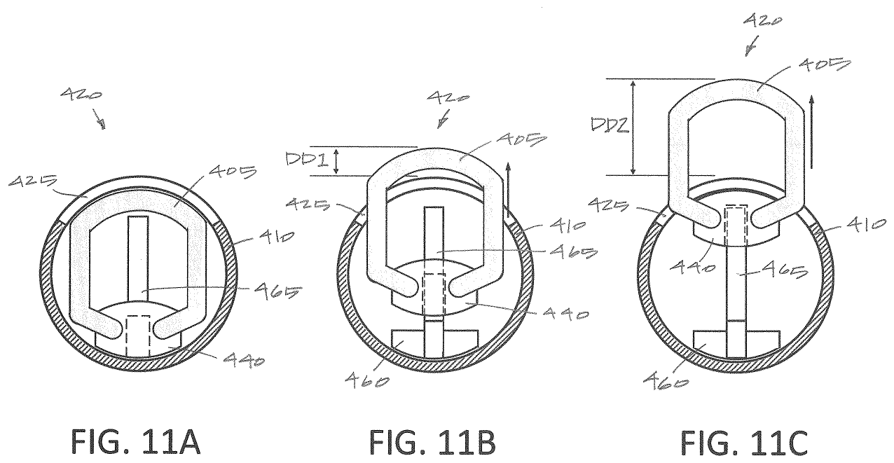

Now, turning to FIGS. 11A to 11C, sectional views of the working end of 420 of the device 400 of FIGS. 8 to 10B are shown with the RF loop electrode 405 in various positions. In FIG. 11A, the loop electrode 405 is in a constrained position within the envelope of the introducer member 410. The loop electrode 405 and electrode shaft 440 are urged into this initial constrained position by a leaf spring or other spring means (not shown) within the passageway 446 of the introducer member 410. FIG. 11B shows the cam surface 465 after being moved distally, which causes the electrode shaft 440 and loop electrode 405 to move outwardly to provide a selected cutting depth indicated by a DD1 that can range from 1 millimeter to 4 mm as described previously. FIG. 11C shows the cam surface 465 moving the electrode shaft 440 and loop electrode 405 outwardly for a maximum cutting depth DD2, which can range from 3 mm to 10 mm.

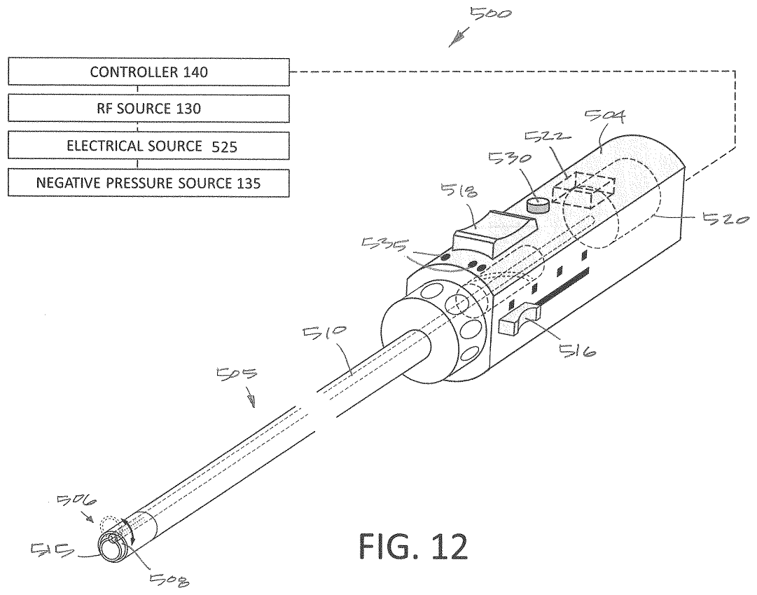

Now, turning to FIG. 12, another variation of a resecting device 500 is shown, which is similar to the device 100 of FIGS. 1 and 2, except it can be operated in first and second modes comprising a manual mode and a controller-assisted mode, respectively. The device 500 has a handle 504 coupled to an introducer member 505 extending to a working end 506. The introducer 505 has an interior passageway 508 carrying a rotatable electrode shaft 510, as described in previous variations. A distal RF loop electrode 515 is rotatable outward from the introducer member 505, as described in previous variations. In this variation, the manual mode of operation is the same as described above in the device of FIG. 1, with the drive assembly comprising a sliding grip 516 operable to move the loop electrode 515 outwardly to provide a selected cutting depth (see FIGS. 3B-3D). The mechanism of the sliding grip 516 includes electrical contacts (not shown), which are coupled to the controller 140 to signal the selected cutting depth to the controller for purposes described below relating to the second controller-assisted mode of operation. After selecting a cutting depth, the physician then would resect targeted tissue by (i) pressing the actuator button 518 to energize the loop electrode 515, (ii) then cutting a path into tissue and then translating the loop electrode through the targeted tissue to cut a strip of tissue, and (iii) then lifting the still-energized loop electrode 515 outwardly to thereby resect and mobilize the tissue strip (see FIGS. 7B-7D).

In the second controller-assisted mode, a motor drive 520 is used to rotate the loop electrode 515. As can be seen in FIG. 12, the handle 504 carries motor drive 520 that is coupled to the rotatable electrode shaft 510 and is configured to rotate the electrode shaft and loop electrode 515 in response to signals from an accelerometer 522. FIG. 12 shows accelerometer 522 carried in the handle 504, which is coupled to the controller 140. The controller 140 is also operatively coupled to an electrical source 525 that powers the electric motor drive 520. Referring to FIG. 12, the handle 504 has a switch 530 that allows the physician to toggle between the first and second modes of operation. The handle 504 further carries LEDs 535 that visually indicate selection of the first or second mode of operation.

In a variation, the accelerometer 522 sends signals to the controller 140 relating to movement of the device 500 or working end 506 during a resection procedure, wherein the controller 140 is configured with algorithms to determine any form of movement of the device 500, such as starting or stopping movement, or rate of change of movement of the device and the loop electrode 515. Responsive to such signals from the accelerometer 522, the controller 140 can change an operating parameter such a power delivered from RF source 130 to the loop electrode 515 and/or rotational movement of the loop electrode provided by the motor drive 520.

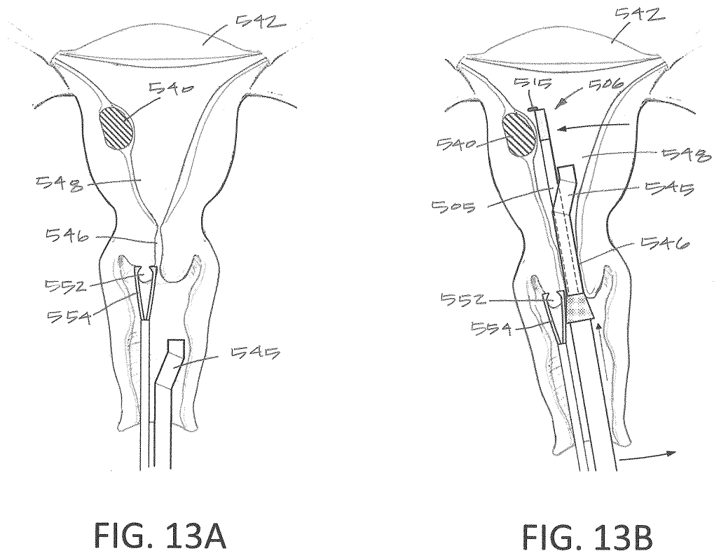

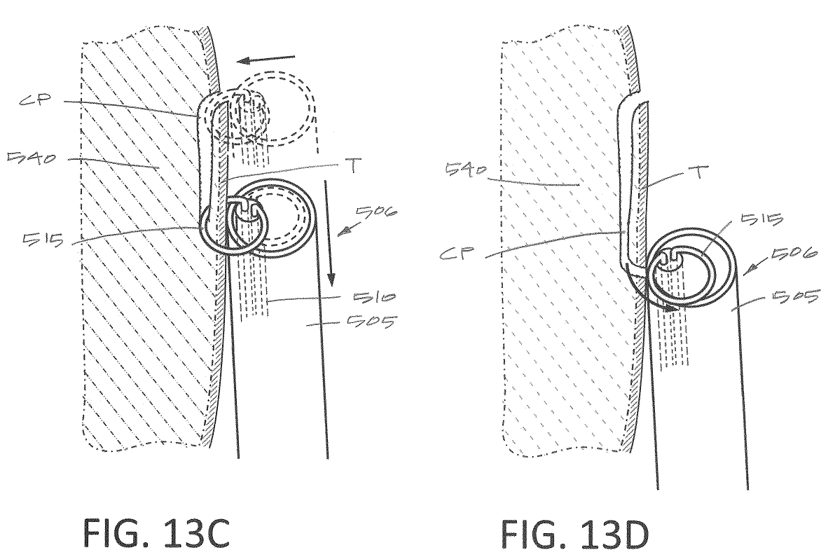

Now, turning to FIGS. 13A to 13D, the controller-assisted mode of operation, is illustrated in a gynecology procedure to resect a fibroid 540 in a patient's uterus 542. A variation of a method is shown using accelerometer 522 and controller 140 to assist in mobilizing a strip of tissue T (FIGS. 13C-13D). In the device of FIGS. 12 and 13A, the introducer member 505 does not include an articulating section as in previous variations. This is for convenience only, and the introducer member 505 optionally has such an articulating section. In FIGS. 13A to 13D, a resecting device 500 with a straight, rigid introducer member 505 is suitable for resecting a fibroid, and an articulating working end is typically not needed.

In FIG. 13A, the physician is preparing to introduce an endoscope 545 through the patient's cervical canal 546 into the uterine cavity 548, with the cervix 552 being stabilized with a tenaculum 554. The endoscope 545 is of the type having an expandable working channel as disclosed in commonly-owned U.S. Pat. No. 11,432,717 titled “Endoscope and Method of Use” authored by Truckai et al. FIG. 13B shows the endoscope 545 after introduction into the uterine cavity 548 with the resecting device 500 advanced through a working channel of the endoscope 545. FIG. 13B further illustrates the working end 506 of resecting device 500 being angled toward the fibroid 540, and the physician also adjusts the sliding grip 516 (FIG. 12) to select a cutting depth of the loop electrode 515. The physician has actuated the switch 530 in the handle 504 to operate in the second controller-assisted mode (FIG. 12).

Now, turning to the enlarged view of FIG. 13C, the physician energizes the RF loop electrode 515 by pressing power button 518 and cuts into the fibroid 540, or adjacent tissue, creating an inward cutting path CP and then translates the working end 506 proximally with the introducer surface in contact with tissue to extend the cutting path CP at the selected cutting depth through or under the fibroid 540 cutting a strip of tissue T. At the time the physician presses the power button 518, the controller logs signals from the accelerometer 522 which indicate movement of the working end 506 and loop electrode 515. A controller algorithm can determine that the working end 506 is being translated axially relative to the axis of the resecting device 500 and endoscope 545 (FIG. 13B). FIG. 13D then illustrates that the physician has cut an elongated cutting path CP at the selected depth and then stops movement of the working end 506. The control algorithm then in response to signals from the accelerometer 522 that movement of the electrode 515 has stopped, actuates the motor drive 520 to rotate the loop electrode 515 inwardly to cut off the end 550 of the tissue strip T. In this mode of operation, the loop electrode 515 remains energized as it moves to an inward position of FIG. 13D to cut off the tissue strip, and the controller algorithm then turns off the power to the electrode, and further the motor drive 520 rotates the de-energized loop electrode outwardly to the previously selected cutting depth. After these sequential steps, the working end 506 and loop electrode 515 are ready to repeat the cutting sequence described above to resect additional tissue strips to complete the resection of fibroid 540.

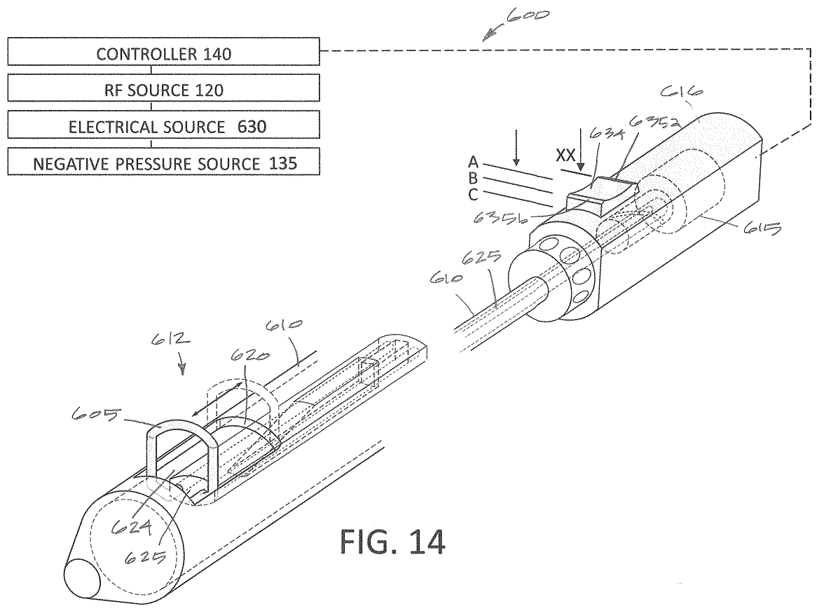

Now, turning to FIG. 14, another variation of a resecting device 600 is illustrated that is similar to device 400 of FIGS. 8 to 10B, except that the controller 140 is adapted to control an automated mechanism to reciprocate an RF loop electrode 605 and move the loop electrode 605 inwardly and optionally outwardly in the introducer member 610 of the device. This variation has an advantage in a method of use wherein the working end 612 is pressed against targeted tissue and held stationary while the loop electrode 605 is moved by a motor drive 615 to resect a strip of tissue in a partly automated manner. The method of using the previous variations typically required the physician to move or translate the loop electrode through tissue to resect a tissue strip.

More in particular, referring to FIG. 14, the resecting device 600 has a handle 616 coupled to introducer member 610 that extends to the working end 612 with an elongated window 620 therein. The loop electrode 605 reciprocates and moves outwardly and inwardly from window 620. The introducer 610 has an interior passageway 624 carrying an electrode shaft 625 of the same type as in device 400 of FIGS. 8 and 9. The electrode shaft 625 and loop electrode 605 of FIG. 14 also is actuated by the same cam mechanism as fully described above and shown in FIGS. 8 and 9. The handle 616 carries the motor drive 615 powered by electrical source 630 that is configured to move the assembly of the electrode shaft 625, cam mechanism, and loop electrode 605 to resect strips of tissue. The motor drive 615 has an output shaft coupled to linear drive and rotation mechanisms known in the art to move the electrode shaft assembly together with the cam mechanism to axially translate the entire loop electrode assembly.

In a variation, an actuator button 634 in the handle 616 is configured to perform multiple functions, including selecting the cutting depth of the loop electrode 605. In FIG. 14, the proximal end 635a of the actuator button 634 can be depressed multiple times to toggle through a plurality of cutting depths that are adjusted by the motor drive 615, for example, from two to five different selected cutting depths of 1 mm to 5 mm. A plurality of LED's can be provided (not shown) on the handle 616 to indicate the selected cutting depth, while the physician can also observe the cutting depth of the loop electrode endoscopically. In a non-deployed or default position, the loop electrode 605 and electrode shaft 625 are positioned distally and inward relative to the window 620.

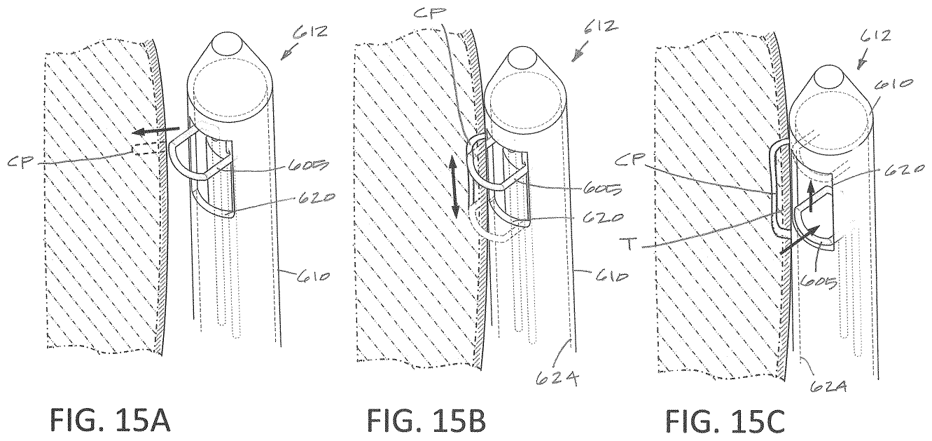

Turning to FIGS. 15A to 15C, a method of use is illustrated wherein the working end 612 is positioned in a saline-distended working space WS. In FIG. 15A, the physician uses the actuator button 634, as described above, to select a cutting depth. Thereafter, the physician depresses the distal end 635b of the actuator button 634 from A to B, which moves the loop electrode 605 outwardly to the selected cutting depth and energizes the loop electrode (FIG. 14). The physician then moves the loop electrode 605 into tissue to cut a cutting path CP as indicated by the dashed line in FIG. 15A until the outer surface of the introducer contacts tissue, as depicted in FIG. 15B.

FIG. 15B illustrates a subsequent step of the method where the physician depresses the distal end 635b of the actuator button 634 from B to C, which signals to controller 140 to actuate the motor drive 615 to move the loop electrode 605 proximally to extend the cutting path CP at the selected cutting depth. FIG. 15C illustrates a continued cutting step wherein the motor drive 615 moves the loop electrode 605 inwardly into the window 620 of introducer 610 to fully resect and mobilize the tissue strip T as the loop electrode 605 reaches its retracted, proximal position. Typically, the strip of tissue T would be aspirated into the internal passageway 624 by the negative pressure source 135 and extracted to a tissue trap (not shown) as is known in that art. FIG. 15C further illustrates that the controller 140 would (i) de-energize the loop electrode 605 when the electrode reaches its retracted, proximal position and then (i) operate the motor drive 615 to move the loop electrode 605 to its distal default position. Thereafter, the physician repeats the above steps to resect additional tissue strips to complete a procedure.

The resecting device 600 of FIG. 14 also can be used a controller 140 configured to operate the loop electrode 605 in a more fully automated manner. For example, the system could be configured so that the physician positions the working end 612 and window 620 against the targeted tissue and then press the actuator button 634 to initiate the following steps: (i) the controller 140 energizes the loop electrode 605, (ii) the motor drive is then activated so the cam mechanism moves the loop electrode 605 outward from the window 615 to cut a cutting path transversely into the tissue to the selected cutting depth, (iii) the motor drive 615 is then activated to cause axial translation of the loop electrode assembly to extend the cutting path axially within the tissue, (iv) the motor drive then retracts the loop electrode 605 inward into the window 615 to cut off the strip of tissue; and (v) the electrode is de-energized, and motor drive returns the loop electrode to the default, distal position. Thereafter, the above steps can be repeated in the same sequence. In this method, pressing the actuator button 634 could initiate a single sequence of steps as described above or simply continue the series of steps until the power button is released. This method would allow that physician to resect multiple strips of tissue without having to translate the resecting device inward and outward of the working channel in the endoscope.

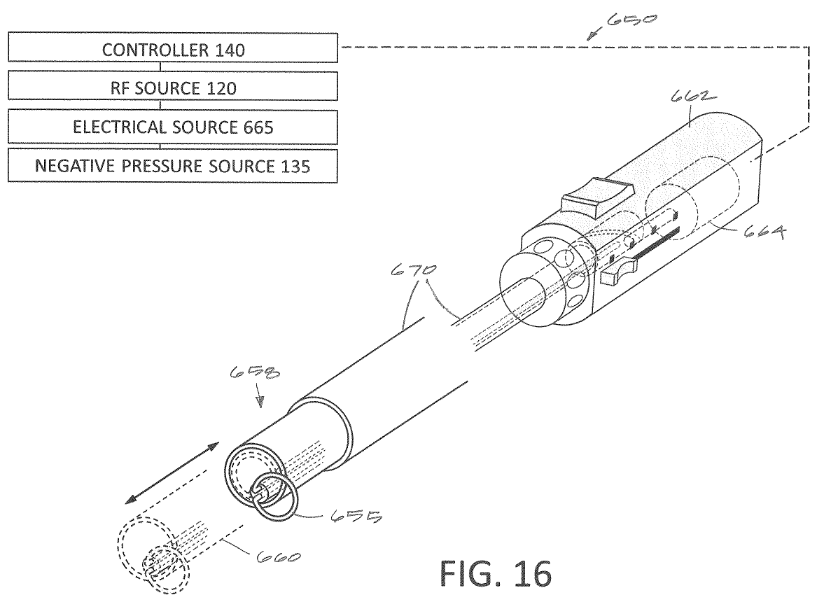

FIG. 16 illustrates another variation of a resecting device 650, which is similar to the device 100 of FIGS. 1 to 3D with an RF loop electrode 655 that can be rotated outwardly and inwardly from the working end 658 relative to the envelope of the introducer assembly 660. The variation or FIG. 16 includes a handle 662 carrying a motor drive 664 that is powered by electrical source 665. In this variation, the motor drive 664 is adapted to reciprocate the entire working end 658 in an outer sleeve 670 of the introducer assembly 660. This variation has an advantage in a method similar to that of device 600 of FIGS. 14-15, wherein physician can move the loop electrode 655 into and out of a tissue surface without manually moving the working end 658 axially. Instead, the variation of FIG. 16 uses the motor drive 664 to reciprocate the working end 658 and loop electrode 655. In this method of use, the physician can maintain the device axially motionless relative to the endoscope (cf. FIGS. 13C-13D), which is an advantage. In this variation, the selected cutting depth is adjusted by the sliding grip 672, as described previously. In a variation, the reciprocation mechanism in the handle 662 can be a form of linear actuator known in the art with a reversible motor drive.

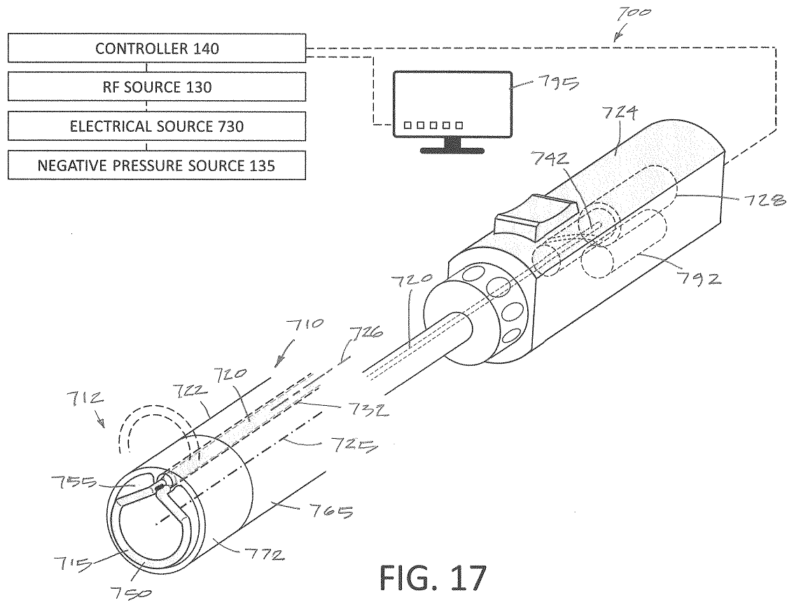

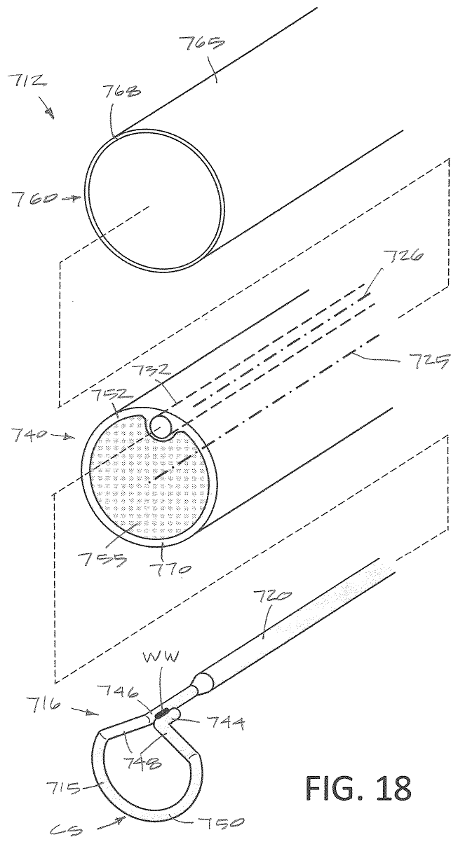

Now, turning to FIGS. 17 and 18, another variation of a resecting device 700 is illustrated that is similar to device 100 of FIGS. 1 to 3D, except that the introducer member 710, working end 712, and RF loop electrode 715 are designed to maximize the cutting depth of the loop electrode 715 when deployed relative to the diameter of the introducer member. As can be seen in FIG. 18, a unitary electrode member 716 comprises loop electrode 715 at the distal end of an electrode shaft 720 that extends through the introducer member 710. The increased cutting depth of the loop electrode 715 is attained by reducing the diameter of the electrode shaft 720 and positioning the electrode shaft 720 close to the introducer envelope 722, which is defined by the outer surface of the introducer member 710. The method of using the resecting device 700 is similar to the methods described above.

More, in particular, FIG. 17 illustrates the resecting device 700 with a handle 724 coupled to introducer member 710 that extends about longitudinal axis 725 to the working end 712. The loop electrode 715 is shown in FIG. 17 in a default or inward position for introduction into a working space and is adapted for deployment outwardly from the introducer member 710 and introducer envelope 722 by rotation of the electrode shaft 720 about its axis 726 as described previously. The deployment mechanism comprises a motor drive 728 carried within the handle 724 configured to rotate the electrode shaft 720, but manual deployment is also possible, as in previous variations.

The motor drive 728 in the handle is powered by electrical source 730 that is similar to previous variations. The resecting device 700 and motor drive 728 are again operatively connected to an RF source 130, negative pressure source 135, and controller 140 that function as described in previous variations.

Now referring to FIG. 18, the components of the introducer member 710 and working end 712 are shown in exploded view. In this variation, the electrode member 716 is configured with loop electrode 715 that extends distally from electrode shaft 720. FIGS. 17 and 18 show the electrode shaft 720 extending through a channel 732 in an extruded central core sleeve 740 of the introducer member 710. The electrode shaft 720 extends through channel 732 to the handle 724, where a proximal end 742 of the electrode shaft is operatively coupled to the motor drive 728 (FIG. 17). In a variation shown in FIG. 18, the electrode member 716 can comprise a tungsten wire with shaft 720 having a first diameter and the distal loop electrode 715 having a second reduced diameter. In a variation, the introducer member 710 is elongated for introduction through a working channel of an endoscope, and it can be understood that the electrode shaft 720 is required to resist twisting and, therefore, has a suitable torque-resistant diameter which is greater than 0.015″ and often greater than 0.018″. In this variation, the tungsten wire portion comprising the loop electrode 715 has a diameter less than 0.015″ and often less than 0.012″. The unitary electrode member 716, as shown in FIG. 18 can be fabricated by providing a suitable tungsten wire or other suitable metal wire having the diameter of the electrode shaft 720 described above and then grinding a distal portion of the wire to a dimension described above for a loop electrode 715 followed by forming the circular shape of the loop electrode 715.

In the variation of FIG. 18, the reduced diameter of the loop electrode 715 reduces the active electrode surface area and thus reduces the power requirements from RF source 130 to ignite a plasma for cutting tissue. In the variation shown in FIG. 18, the loop electrode 715 extends in 360°, and a weld WW fuses the tail end 744 of the loop with initial section 746 of the loop. It can be seen that in FIG. 18 that the loop electrode 715 has a cylindrical shape CS at least in part and leg portions 748 that extend to axial the portion of the electrode shaft 720. The circular shape CS extends from 180° to 360° around the loop shape and comprises the active electrode 750. The leg portions 748 of electrode 715 may be covered with an insulative material such as an epoxy or heat shrink material (not shown) to further reduce the active electrode surface area. In a typical variation, the bare wire portion of loop electrode 715 comprising the active electrode 750 extends at least 180° around the circular shape CS of the loop.

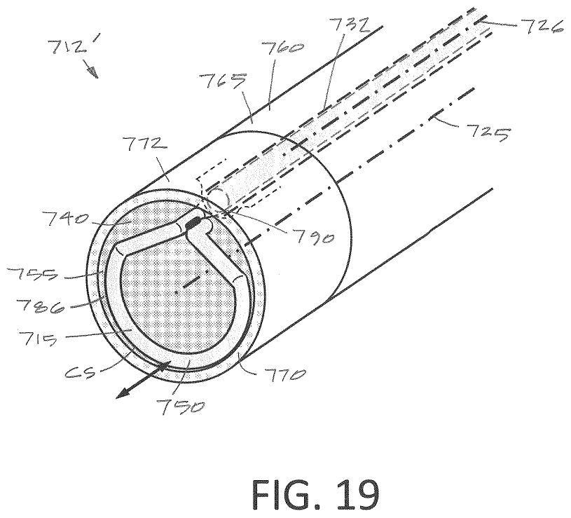

Referring to FIG. 18, the central core sleeve 740 of the introducer member 710 comprises an extruded polymeric material such as FEP. The diameter of channel 732 in the wall 752 of the core sleeve 740 is dimensioned to allow rotation and optional axial movement of the electrode shaft 720 therein. The central passageway 755 in the core sleeve 740 communicates with the negative pressure source 135. In FIGS. 18 and 19, it can be understood that the combination of small diameter electrode shaft 720 and extruded core sleeve 740 allows for an increased cutting depth when the loop electrode 715 is rotated 180° out of its default inward position of FIG. 17.

Referring again to FIG. 18, the extruded core sleeve 740 is disposed within a thin metal outer sleeve 760, such as stainless steel, that provides stiffness for the assembly comprising the elongated introducer member 710. In this variation, the introducer member 710 is configured as a rigid assembly and is not adapted for articulation as in some previous variations. The metal outer sleeve 760 can comprise a stainless steel tube with a wall thickness of less than 0.006″ or less than 0.005″ to provide a suitable stiffness to the introducer in a variation where the introducer has an outer diameter of less than 6 mm or less than 5 mm. In the variation of FIGS. 17 to 19, the outer sleeve 760 comprises a return electrode 765. In an assembled variation shown in FIG. 19, the outer sleeve 760 has a distal end 768 that is spaced apart from the distal end 770 of the core sleeve 740 to provide an insulative material between the loop electrode 715 and the return electrode 765 during use. As can be seen in FIGS. 17, 19, and 22, an insulative circumferential band 772 is provided around the distal end 770 of the core sleeve 740, which can comprise a heat shrink material, epoxy, or the like. The axial length of the insulative circumferential band 772 is at least 1.0 mm or at least 2.0 mm and often greater than 4.0 mm.

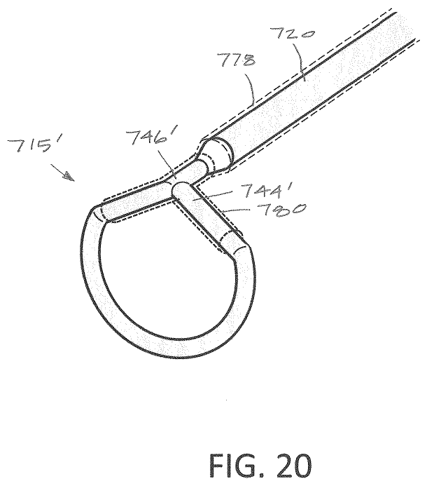

FIG. 20 illustrates a variation of loop electrode 715′ wherein the tail end 744′ of the loop is not welded to the initial loop section 746′ as in the variation of FIG. 18. In FIG. 20, the tail end 744′ and initial section 746′ are bonded together with an epoxy material 780 molded around a portion of electrode 715′ to provide an insulative layer and to connect the tail end 744′ and initial section 746′ of the loop electrode. In this variation, the electrode shaft 720 is also shown encased in a heat shrink member 778 to add to the insulative properties of the electrode shaft 720.

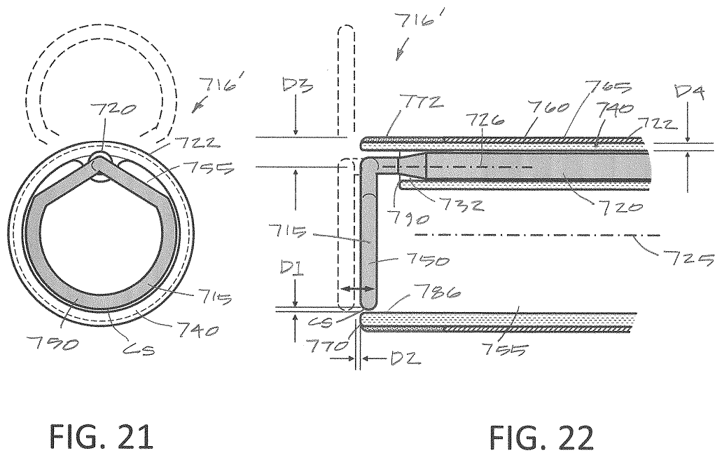

Now, turning to FIGS. 19, 21, and 22, another aspect of the working end 712′ of a resecting device is shown. In this variation, the working end 712′ includes a loop electrode 715 configured for axial extension and retraction in the central passageway 755 of the working end, wherein the outer surface 785 of the circular shape CS of loop electrode 715 is closely matched in dimension to the surfaces 786 of passageway 755 so that when the loop electrode 715 is retracted into the passageway 755, any eschar sticking to the loop electrode 715 will be scraped away by surface 786. FIG. 22 shows the radial distance D1 between the loop electrode 715 and surface 786 is minimal and is often less than 0.5 mm. Similarly, as can be seen in FIGS. 21 and 22, when the loop electrode 715 is extended outwardly from the passageway 755, rotation of the loop electrode from any deployed position to the default, inward position will scrape eschar from the proximal surface 788 of the loop electrode 715. FIG. 22 shows the axial distance D2 between the loop electrode 715 and distal end 770 of the core sleeve 740 is minimal and is often less than 0.5 mm. As can be seen in FIGS. 19 and 22, the distal end 790 of channel 732 carrying the electrode shaft 720 is recessed in the core sleeve 740 to allow retraction of the loop electrode 715 into the passageway 755.

Referring back to FIG. 17, it can be seen that the handle 724 carries a second motor drive 792 that is adapted for causing axial movement of the loop electrode 715 as shown in FIGS. 19 and 22. Thus, in a variation, the handle 724 carries the first motor drive 728 for rotational deployment of the loop electrode 715, and the second motor drive 792 is configured for the axial extension and retraction of loop electrode 715 from the passageway 755. In other variations, a single motor can have mechanisms adapted for performing both rotational and axial movements.

In a variation, the motor drives 728 and 792 comprise encoder motors, which send signals to the controller 140 as to the degree of rotation of the motor shaft, which in turn can be used to determine the dimension of axial deployment and outward deployment of the loop electrode 715 which in turn determines the cutting depth. In the variation shown in FIG. 17, the controller 140 is also coupled to a touch screen display 795 where the physician can select operating parameters of the systems, for example, the selected cutting depth. In this variation, the actuator button 796 in the handle 724 is adapted only for activating the loop electrode 715 while the touch screen display 795 is used for selecting other operational parameters.

In the cut-away view of FIG. 22, the dimensions of components of the working end 716′ are shown that provide for maximizing the cutting depth of the loop electrode 715. As can be understood, referring to FIG. 22, the objective is to position the rotational axis 726 of the electrode shaft 720 as close as possible to the outer surface of the introducer 710 that defines introducer envelope 722 indicated at D3, which is less than 0.010″ or less than 0.050″. However, the outer sleeve 760 comprises return electrode 765, so that suitable insulative components must separate the electrode shaft 720 from the outer sleeve 760 to prevent electrical coupling within the wall of the introducer 710. In a variation, the dialectic material in FIG. 22 has a dimension D4 of at least 0.003″ or at least 0.005″ extending through the introducer member 710 that comprises the wall thickness of the core sleeve 740 around the channel 732 and optionally the thickness of a heat shrink material 778 encasing the electrode shaft 720 (see FIG. 20). The resection devices described above can be used in various surgical fields such as urology, gynecology, arthroscopy, and other fields where resecting procedures are performed in a saline-immersed working space, such as in FIGS. 7A-7D and 13A-13D. For example, the RF devices and methods described above can be used in a BPH procedure to resect tissue to reduce the volume of a patient's prostate. In such a procedure, an articulating working end may not be needed, and a straight, rigid introducer may be used. Small-diameter RF devices, as described above, can be used in the field of arthroscopy, for example, to resect tissue in a patient's shoulder, hip, or knee again without an articulating working end.

In the variations described above, the negative pressure source 135 (FIGS. 1 and 8) communicates with a passageway in the introducer wherein the fluid inflow port is either at the distal end of the introducer or in a slot in the introducer from which the RF loop electrode is extended. It should be appreciated that the controller 140, or a fluid management system, typically will be used to control fluid pressure in the working space. Thus, the openings or ports in the working end of the introducer can be dimensioned to accommodate the required fluid flows, and additional ports may be disposed on any side of the working end to provide for adequate fluid outflows, wherein fluid inflows typically flow an inflow channel in the endoscope FIG. 7A).

Although particular variations of the present invention have been described above in detail, it will be understood that this description is merely for purposes of illustration, and the above description of the invention is not exhaustive. The variations above are shown with a moto drive for moving a cutting sleeve helically and rotationally, but manually operated mechanisms are also possible for either or both such helical and rotational movements. Specific features of the invention are shown in some drawings and not in others, and this is for convenience only, and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Various changes may be made to the invention described, and equivalents (whether recited herein or not included for the sake of some brevity) may be substituted without departing from the true spirit and scope of the invention. Also, any optional feature of the inventive variations may be set forth and claimed independently, or in combination with any one or more of the features described herein. Accordingly, the invention contemplates combinations of various aspects of the embodiments or combinations of the embodiments themselves, where possible. Reference to a singular item includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments, or the embodiments themselves can be combined. Where such combinations are intended to be within the scope of this disclosure.

1. An electrosurgical device, comprising:
a handle coupled to an introducer member extending about a central axis to a working end, wherein the introducer member has an outer surface that defines an introducer envelope extending axially from the introducer member;
an electrode shaft moveably located within the introducer member;
an RF loop electrode carried by the electrode shaft and positioned at the working end extending in a transverse plane that is from 45 to 90 degrees transverse to the central axis;
a drive assembly within the handle and coupled to the electrode shaft, where the drive assembly is configured to move the RF loop electrode between a first position within the introducer envelope for introduction into a working space and a second position radially outward from the introducer envelope for resecting tissue, the second position defining a selected cutting depth;
wherein the tissue is resected by translating the working end over the target tissue while the RF loop electrode remains in the second position such that the RF electrode cuts a tissue strip at the selected cutting depth.

2. The electrosurgical device of claim 1, wherein the RF loop electrode is carried at a distal end of the electrode shaft.

3. The electrosurgical device of claim 2, wherein the drive assembly is adapted to move the RF loop electrode between the first position and the second position by at least one of rotation of the electrode shaft and lateral movement of the electrode shaft.

4. The electrosurgical device of claim 3, wherein the drive assembly is further adapted to move the RF loop electrode axially between a distal position and a proximal position relative to the introducer member.

5. The electrosurgical device of claim 4, wherein the drive assembly is actuated by at least one of a motor drive and manual actuation.

6. The electrosurgical device of claim 5, wherein the drive assembly is selected by at least one of an actuator in the handle and an icon on a touch screen.

7. The electrosurgical device of claim 1, wherein the RF loop electrode comprises an inflexible wire.

8. The electrosurgical device of claim 1, wherein the outer surface of the RF loop electrode in the second position is positioned outward of the introducer envelope by at least 0.5 mm.

9. The electrosurgical device of claim 1, wherein the outer surface of the RF loop electrode in the second position is positioned outward of the introducer envelope by at least 2 mm.

10. The electrosurgical device of claim 1, wherein the outer surface of the RF loop electrode in the second position is positioned outward of the introducer envelope by at least 4 mm.

11. The electrosurgical device of claim 5, further comprising an RF source coupled to the RF loop electrode and where the drive assembly includes a motor drive, and a controller configured to control operating parameters of the RF source and motor drive.

12. The electrosurgical device of claim 11, wherein the motor drive comprises an encoder motor configured to send signals to the controller for determining a degree of rotation and corresponding cutting depth of the RF loop electrode when deployed.

13. The electrosurgical device of claim 11, further comprising an accelerometer carried by the electrosurgical device and configured to send signals to the controller relating to movement of the electrosurgical device.

14. The electrosurgical device of claim 1, wherein the introducer member includes an articulating section in the working end that is actuatable from the handle.

15. The electrosurgical device of claim 2, wherein the RF loop electrode comprises an active electrode, a distal circumferential band of the working end comprises an insulative material, and an outer surface of the working end proximal to the distal circumferential band comprises a return electrode.

16. The electrosurgical device of claim 15, wherein a dimension of insulative material between the electrode shaft and the return electrode is at least 0.003″.

17. The electrosurgical device of claim 1, further comprising an extension-retraction mechanism for extending and retracting the RF loop electrode relative to a passageway in the introducer member, wherein an outer surface of the RF loop electrode is dimensioned to match a surface of the passageway wherein retraction of the RF loop electrode scrapes eschar from the RF loop electrode when retracted into the passageway.

18. A method of resecting tissue in a working space in a patient, comprising:
providing an introducer having an outer surface that defines an introducer envelope extending axially from the introducer, the introducer extending about a central axis to a working end carrying an RF loop electrode that is moveable between a first position within the introducer envelope and a second position radially outward from the introducer envelope, the second position defining a selected cutting depth;
introducing the working end in the first position through a working channel of an endoscope into the working space;
moving the RF loop electrode to the second position;
resecting the tissue by activating the RF loop electrode and translating the working end over the tissue while the RF loop electrode remains in the second position such that the RF electrode cuts a tissue strip at the selected cutting depth; and
moving the RF loop electrode from the second position to the first position thereby mobilizing the tissue strip.

19. The method of claim 18, wherein the RF loop electrode extends in a transverse plane that is from 45 to 90 degrees transverse to a central axis of the introducer in the second position.

20. The method of claim 18, wherein the selected cutting depth is selected by a mechanism in a handle portion of the introducer.

21. The method of claim 18, wherein the RF loop electrode is moved between the first position and the second position in a rotational direction relative to the central axis of the introducer.

22. The method of claim 18, wherein the RF loop electrode is moved between the first position and the second position in a transverse direction relative to the central axis of the introducer.

23. The method of claim 18, wherein the RF loop electrode is moved between the first position and the second position by at least one of a motor drive and manual actuation.

24. The method of claim 18, wherein the working end is translated over tissue by at least one of a motor drive and manual actuation.

25. The method of claim 18, wherein a controller is configured to control movement of the RF loop electrode.

26. The method of claim 25, further comprising sending signals of movement of the introducer or the RF loop electrode to the controller using an accelerometer, and controlling movement of the RF loop electrode responsive to the signals using the controller.
